(19) United States
(12) Patent Application Publication
(10) Pub. No.: US 2010/0170148 A1
(43) Pub. Date: Jul. 8, 2010

(54) Host Cells and Methods for Producing Fatty Acid Derived Compounds

(75) Inventors: Eric J. Steen, Berkeley, CA (US); Jay D. Keasling, Berkeley, CA (US)

Correspondence Address: LAWRENCE BERKELEY NATIONAL LABORATORY, Technology Transfer & Intellectual Propery Managem, One Cyolotron Road MS 56A-120, BERKELEY, CA, 94720, US

(73) Assignee: The Regents of the University of California, Oakland, CA (US)

(21) Appl. No.: 12/643,817

(22) Filed: Dec. 21, 2009

Related U.S. Application Data
(63) Continuation of application No. PCT/US2008/068833, filed on Jun. 30, 2008.
(60) Provisional application No. 60/947,332, filed on Jun. 29, 2007.

(51) Int. Cl. C10L 5/00; C12P 7/24; C12N 1/21; C12N 5/10; C12N 1/13; C12N 1/15; C12N 1/19

(52) U.S. Cl. 44/605; 435/147; 435/252.3; 435/252.31; 435/252.33; 435/252.34; 435/325; 435/348; 435/257.2; 435/254.11; 435/254.2

(57) ABSTRACT

The present invention provides for a method of producing one or more fatty acid derived compounds in a genetically modified host cell which does not naturally produce the one or more derived fatty acid derived compounds. The invention provides for the biosynthesis of fatty acid derived compounds such as C18 aldehydes, C18 alcohols, C18 alkanes, and C17 alkanes from C18-CoA which in turn is synthesized from butyryl-CoA. The host cell can be further modified to increase fatty acid production or export of the desired fatty acid derived compound, and/or decrease fatty acid storage or metabolism.

### CROSS-REFERENCES TO RELATED APPLICATIONS

This application claims benefit as a continuation application of PCT International Application No. PCT/US2008/68833, filed Jun. 30, 2008, which claims priority to U.S. Provisional Application Ser. No. 60/947,332, filed Jun. 29, 2007, the disclosures of which are incorporated by reference in their entireties.

### STATEMENT AS TO RIGHTS TO INVENTIONS MADE UNDER FEDERALLY SPONSORED RESEARCH OR DEVELOPMENT

The invention described and claimed herein was made utilizing funds supplied by the U.S. Department of Energy under Contract No. DE-AC02-05CH11231. The government has certain rights in this invention.

### FIELD OF THE INVENTION

The present invention is in the field of production of fatty acid derived compounds, and in particular host cells that are genetically modified to produce fatty acid derived compounds.

### BACKGROUND OF THE INVENTION

Petroleum derived fuels have been the primary source of energy for over a hundred years. Petroleum, however, has formed over millions of years in nature and is not a renewable source of energy. A significant amount of research in alternative fuels has been ongoing for decades. Within this field, ethanol has been studied intensively as a gasoline substitute and the use of ethanol as transportation fuel has been increasing recently (Gray et al., Curr Opin Chem Biol 2006, 10:141). However, the efficiency of ethanol as a fuel is still in debate (Pimentel, Natural Resources Research 2005, 14:65; Farrell et al., Science 2006, 311:506). There is interest to design several potential alternative fuel molecules other than ethanol, which can be produced biosynthetically, and to develop the biosynthetic pathways for enhanced production of the target fuel molecules using synthetic biology.

This present invention involves the biosynthesis of fatty acid derived molecules which can be a source of renewable fuels, therapeutic compounds, and expensive oils.

### BRIEF SUMMARY OF THE INVENTION

The present invention provides for a method of producing one or more fatty acid derived compounds in a genetically modified host cell which does not naturally produce the one or more derived fatty acid derived compounds. The invention provides for the biosynthesis of fatty acid derived compounds such as C18 aldehydes, C18 alcohols, C18 alkanes, and C17 alkanes from C18-CoA which in turn is synthesized from butyryl-CoA. Such host cells are either naturally capable of producing C18-CoA or genetically modified to express enzymes capable of synthesizing C18-CoA.

The present invention also provides for a method of producing C18-CoA in a genetically modified host cell which does not naturally produce C18-CoA. The host cells are modified to express enzymes capable of synthesizing C18-CoA from butyryl-CoA. Such host cells are either naturally capable of producing butyryl-CoA or genetically modified to express enzymes capable of synthesizing butyryl-CoA.

The present invention also provides for a method of producing a fatty acid derived compound in a genetically modified host cell that is modified by the increased expression of one or more genes involved in the production of fatty acid compounds; such that the production of fatty acid compounds by the host cell is increased. Such gene encode following proteins: acetyl carboxylase (ACC), cytosolic thiosterase (teas), and acyl-carrier protein (AcpP).

The present invention also provides for a method of producing a fatty acid derived compound in a genetically modified host cell that is modified by the decreased or lack of expression of one or more genes encoding proteins involved in the storage and/or metabolism of fatty acid compounds; such that the storage and/or metabolism of fatty acid compounds by the host cell is decreased. Such genes include the following: the are1, are2, dga1, and lro1 genes.

The present invention also provides for a method of producing a fatty acid derived compound in a genetically modified host cell that is modified to express or have increased expression of an ABC transporter that is capable of exporting or increasing the export of any of the fatty acid derived compounds from the host cell. Such an ABC transporter is the plant Cer5.

The present invention further provides for a genetically modified host cell useful for the methods of the present invention. The host cell can be genetically modified in any combination of the one or more genetic modifications described herein.

The present invention further provides for an isolated fatty acid derived compound produced from the method of the present invention.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows the biosynthetic pathway for producing fatty acid derived compounds from butyryl-CoA. An enzyme capable of catalyzing each reaction is shown (with the corresponding Genbank accession number).

FIG. 2 shows the biosynthetic pathway for producing butyryl-CoA from acetyl-CoA. An enzyme capable of catalyzing each reaction is shown.

FIG. 3 shows a fatty acid and long-chain alcohol biosynthesis pathway for S. cerevisiae.

FIG. 4 shows fatty acid levels in E. coli in which a cytosolic esterase has been overexpressed.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The terms “host cell” and “host microorganism” are used interchangeably herein to refer to a living biological cell that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host microorganism, wherein the expression vector contains a nucleic acid sequence coding for an enzyme that is not normally found in a host microorganism. With reference to the host microorganism's genome, then, the nucleic acid sequence that codes for the enzyme is heterologous.

The terms “expression vector” or “vector” refer to a compound and/or composition that transduces, transforms, or infects a host microorganism, thereby causing the cell to express nucleic acids and/or proteins other than those native to the cell, or in a manner not native to the cell. An “expression vector” contains a sequence of nucleic acids (ordinarily RNA or DNA) to be expressed by the host microorganism. Optionally, the expression vector also comprises materials to aid in achieving entry of the nucleic acid into the host microorganism, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host microorganism and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host microorganism or cell. Only when the sequence of nucleic acids becomes stably replicated by the cell does the host microorganism or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

The terms “isolated” or “biologically pure” refer to material that is substantially or essentially free of components that normally accompany it in its native state.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., arninoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

In some embodiments of invention, the invention provides for a method for producing a C18 aldehyde in a genetically modified host cell, the method comprising: culturing a genetically modified host cell under a suitable condition, wherein the genetically modified host cell comprises a first enzyme capable of converting a C18-CoA to a C18 aldehyde and optionally a C18 alcohol, and optionally a second enzyme capable of converting the C18 aldehyde to a C17 alkane or a third enzyme capable of converting the C18 alcohol to a C18 alkane, such that the culturing results in the genetically modified host cell producing the C18 aldehyde, and optionally the C17 alkane, the C18 alcohol, or C18 alkane, or a combination thereof.

In some embodiments, the genetically modified host cell comprises a first nucleic acid construct encoding the first enzyme, and optionally a second nucleic acid construct encoding the second enzyme and/or third enzyme, and the culturing results in the expression of the first enzyme, and optionally the second enzyme and/or the third enzyme.

In some embodiments, the method further comprises the step of: introducing the first nucleic acid construct encoding the first enzyme, and optionally the second nucleic acid construct encoding the second enzyme and/or third enzyme, into the genetically modified host cell, wherein the introducing step is prior the culturing step.

In some embodiments, the method further comprises the step of recovering the produced C18 aldehyde, or optionally the C17 alkane, the C18 alcohol, or the C18 alkane, or a combination thereof, wherein the recovering step is concurrent or subsequent to the culturing step.

In some embodiments, the method comprises a method of genetically modifying a cell, e.g., a bacterial or yeast cell, to increase expression of one or more genes involved in the production of fatty acid compounds; such that the production of fatty acid compounds by the cell is increased. Such genes encode proteins such as acetyl carboxylase (ACC), cytosolic thiosterase (teas), a fatty acid synthase, and acyl-carrier protein (AcpP). In some embodiments, the genetically modified cell may be modified to produce higher levels of cytosolic acetyl-coA. Thus, in some embodiments a genetically modified cell may comprise a modification to express, or increase expression of proteins such as ATP citrate lyase.

In some embodiments, the genetically modified host cell comprise one or more nucleic acid constructs encoding an enzyme capable of converting butyryl-CoA to C10-CoA, an enzyme capable of converting C10-CoA to C14-CoA; and an enzyme capable of converting the C14-CoA to C18-CoA, such that the culturing results in the genetically modified host cell producing the C18-CoA. In some embodiments, the host cell comprises at least one enzyme selected from the group consisting of Trypanosoma ELO1, ELO2, and ELO3 enzymes. In some embodiments, the genetically modified host cell further comprises a nucleic acid construct that encodes an enzyme that synthesizes butyryl-CoA from acetyl-CoA.

### Enzymes and Constructs Encoding Thereof

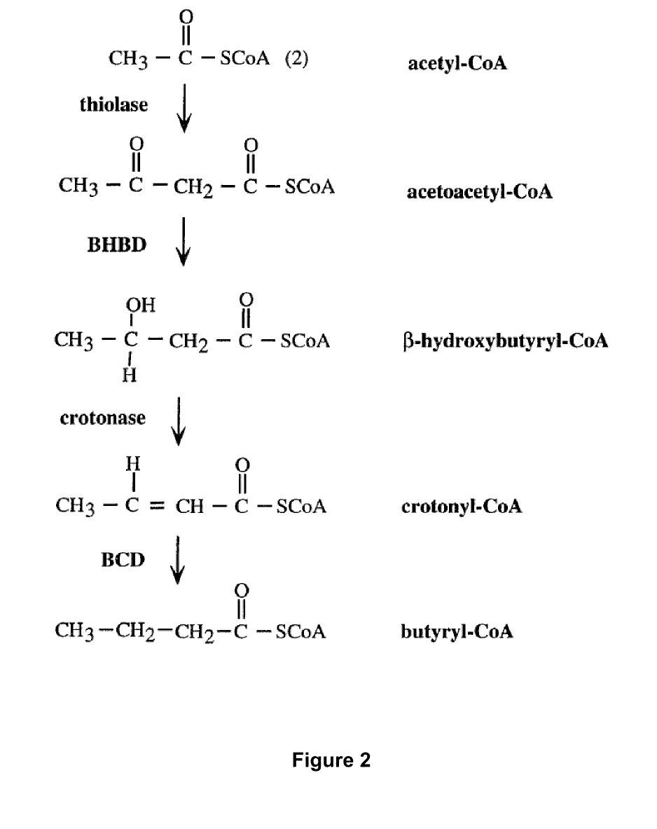

The enzymes capable of synthesizing butyryl-CoA from acetyl-CoA are described by Boynton, Z. L. (Ph.D. Thesis: Characterization of metabolism and genes in the fermentation pathway of Clostridium acetobutylicum ATCC824, UMI, Michigan, 1996; UMI No. 9631057), Boynton et al. (J. Bacteriol. 178(11): 3015-3024, 1996), and Bennett et al. (FEMS Microbiol. Rev. 17(3):241-249, 1995), which are incorporated in their entireties by reference. These enzymes include thiolase (such as acetyl-CoA acetyltransferase), β-hydroxybutyryl-Co dehydrogenase (BHBD; encoded by the hbd gene), crotonase (encoded by the crt gene), and butyryl-CoA dehydrogenase (BCD; encoded by the bcd gene). The pathway in which butyryl-CoA is synthesized from acetyl-CoA is shown in FIG. 2. These genes can be readily cloned from any Clostridium sp., such as Clostridium acetobutylicum. In particular, these genes can be readily cloned from Clostridium acetobutylicum ATCC824 (Boynton et al., 1996).

A suitable enzyme for converting a butyryl-CoA to a C10-CoA is Trypanosoma brucei fatty acid elongase (ELO1) (Genbank accession no. AAX70671). ELO1 has the following amino acid sequence (SEQ ID NO:1):

| | |
|---|---|
| 1 | mfftppqlqk leqdwnglav rdwmianvdv vlyisflylg fvfigpklfa klvgtnpaaa |
| 61 | aagarsadgt gspivrrsmv vwnlalsifs ifgtstvtpv llrnlankgf ygatcdfket |
| 121 | efyttnvgfw mgifalskip elvdtiflvl qgkqelpflh wyhhvtvllf swhtycvgss |
| 181 | ayiwvaamny svhsvmylyf alaalgykrv vrplapyiti iqilqmvvgc yvtifalqel |
| 241 | hgeggrgcgv spanmriqlv myasylylfs kmfvasyirp pkrptvggps stagvsngsv |
| 301 | ekkvk |

A suitable enzyme for converting a C10-CoA to a C14-CoA is Trypanosoma brucei fatty acid elongase (ELO2) (Genbank accession no. AAX70672). ELO2 has the following amino acid sequence (SEQ ID NO:2):

| | |
|---|---|
| 1 | mfpyvtdysg fairkwmidn vdvagflcll ylglvwkgpg vvkslreknl inatllqgvf |
| 61 | imwnlflstf svigmivvvp aaiahisnkg lvpalcerdv nmiydspvgf wvgvfalski |
| 121 | pelfdtvllv lqgkqppflh wyhhttvlif swqsycegss tifvfvamnl tvhavmyfyf |
| 181 | amcasgfkai mrtiapviti mqilqmivgs avtmysayvl ynpqpdgpqt cdvtkasarm |
| 241 | gvvmylsyly lfaalfvesy lkpkkrteks k |

A suitable enzyme for converting a C14-CoA to a C18-CoA is Trypanosoma brucei fatty acid elongase (ELO3) (Genbank accession no. AAX70673). ELO3 has the following amino acid sequence (SEQ ID NO:3):

| | |
|---|---|
| 1 | mlmnfggsyd ayinnfqgtf laewmldhps vpyiagvmyl ilvlyvpksi masqpplnlr |
| 61 | aanivwnlfl tlfsmcgayy tvpylvkafm npeivmaasg ikldantspi ithsgfyttt |
| 121 | caladsfyfn gdvgfwvalf alskipemid taflvfqkkp viflhwyhhl tvmlfcwfay |
| 181 | vqkissglwf asmnysvhsi mylyyfvcac ghrrlvrpfa piitfvqifq mvvgtivvcy |
| 241 | tytvkhvlgr sctvtdfslh tglvmyvsyl llfsqlfyrs ylsprdkasi phvaaeikkk |
| 301 | e |

A suitable enzyme for converting a C18-CoA to a C18 aldehyde is Arabidopsis thaliana cuticle protein (WAX2) (Genbank accession no. AY131334) as disclosed in Chen et al., Plant Cell 15 (5): 1170-1185 (2003), which is incorporated in its entirety by reference. WAX2 is also taught in U.S. Patent Application Pub. No. 2006/0107349, which is incorporated in its entirety by reference. WAX2 has the following amino acid sequence (SEQ ID NO:4):

MVAFLSAWPWENFGNLKYLLYAPLAAQVVYSWVYEEDISKVLWCIHILII
CGLKALVHELWSVFNNMLFVTRTLRINPKGIDFKQIDHEWHWDNYIILQA
IIVSLICYMSPPLMMMINSLPLWNTKGLIALIVLHVTFSEPLYYFLHRSF
HRNNYFFTHYHSFHHSSPVPHPMTAGNATLLENIILCVVAGVPLIGCCLF
GVGSLSAIYGYAVMFDFMRCLGHCNVEIFSHKLFEILPVLRYLIYTPTYH
SLHHQEMGTNFCLFMPLFDVLGDTQNPNSWELQKKIRLSAGERKRVPEFV
FLAHGVDVMSAMHAPFVFRSFASMPYTTRIFLLPMWPFTFCVMLGMWAWS
KTFLFSFYTLRNNLCQTWGVPRFGFQYFLPFATKGINDQIEAAILRADKI
GVKVISLAALNKNEALNGGGTLFVNKHPDLRVRVVHGNTLTAAVILYEIP
KDVNEVFLTGATSKLGRAIALYLCRRGVRVLMLTLSMERFQKIQKEAPVE
FQNNLVQVTKYNAAQHCKTWIVGKWLTPREQSWAPAGTHFHQFVVPPILK
RFFNCTYGDLAAMKLPKDVEGLGTCEYTMERGVVHACHAGGVVHMLEGWK
HHEVGAIDVDRIDLVWEAAMKYGLSAVSSLTN

A suitable enzyme for converting a C18-CoA to a C18 aldehyde is first Bombyx mori fatty-acyl reductase (FAR) (Genbank accession no. AB104896) as disclosed in Moto et al., Proc. Natl. Acad. Sci. USA 100 (16), 9156-9161 (2003), which is incorporated in its entirety by reference. FAR has the following amino acid sequence (SEQ ID NO:5):

MSHNGTLDEHYQTVREFYDGKSVFITGATGFLGKAYVEKLAYSCPGIVSI
YILIRDKKGSNTEERMRKYLDQPIFSRIKYEHPEYFKKIIPISGDITAPK
LGLCDEERNILINEVSIVIHSAASVKLNDHLKFTLNTNVGGTMKVLELVK
EMKNLAMFVYVSTAYSNTSQRILEEKLYPQSLNLNEIQKFAEEHYILGKD
NDEMIKFIGNHPNTYAYTKALAENLVAEEHGEIPTIIIRPSIITASAEEP
VRGFVDSWSGATAMAAFALKGWNNIMYSTGEENIDLIPLDYVVNLTLVAI
AKYKPTKEVTVYHVTTSDLNPISIRRIFIKLSEFASKNPTSNAAPFAATT
LLTKQKPLIKLVTFLMQTTPAFLADLWMKTQRKEAKFVKQHNLVVRSRDQ
LEFFTSQSWLLRCERARVLSAALSDSDRAVFRCDPSTIDWDQYLPIYFEG
INKHLFKNKL

A suitable enzyme for converting a C18-CoA to a C18 aldehyde is a second Bombyx mori fatty-acyl reductase (FAR) (Genbank accession no. AB104897) as disclosed in Moto et al., Proc. Natl. Acad. Sci. USA 100 (16), 9156-9161 (2003), which is incorporated in its entirety by reference. FAR has the following amino acid sequence (SEQ ID NO:6):

MSHNGTLDEHYQTVSEFYDGKSVFITGATGFLGKAYVEKLAYSCPGIVSI
YILIRNKKGSNTEERMRKYLDQPIFSRIKYEHPEYFKKIIPISGDIAAPK
LGLCDEERNILINEVSIVIHSAASVKLNDHLKFTLNTNVGGTMKVLELVK
EMKNLAMFVYVSTAYSNTSQRILEEKLYPQSLNLSEIQKFAEEHYILGKD
DDEMIKFIGNHPNTYAYTKALAENLVAEEHGEIPTIIIRPSIITASAEEP
VRGFVDSWSGATAMAASTLKGWNYIMYSTGEENIDLIPLDYVVNLTLVAI
AKNKPTKEVTVYHVTTSDLNPISIRRIFIKLSEFASKNPTSNAAPFAATT
LLTKQKPLIKLVTFLMQTTPAFLADFWMKTQRKEAKFVKQHNLVVRSRDQ
LEFFPSQSWLLRCERARVLSAGLGDSGRAVFRCDPSPIDWDQYLPIYFEG
INKHLFKNKF

A suitable enzyme for converting a C18-CoA to a C18 aldehyde is Mus musculus male sterility domain containing 2 protein (FAR1) (Genbank accession no. BC007178) as disclosed in Strausberg et al., Proc. Natl. Acad. Sci. USA 99 (26):16899-16903 (2002), which is incorporated in its entirety by reference. FAR1 has the following amino acid sequence (SEQ ID NO:7):

MVSIPEYYEGKNILLTGATGFLGKVLLEKLLRSCPRVNSVYVLVRQKAGQ
TPQERVEEILSSKLFDRLRDENPDFREKIIAINSELTQPKLALSEEDKEI
IIDSTNVIFHCAATVRFNENLRDAVQLNVIATRQLILLAQQMKNLEVFMH
VSTAYAYCNRKHIDEVVYPPPVDPKKLIDSLEWMDDGLVNDITPKLIGDR
PNTYIYTKALAEYVVQQEGAKLNVAIVRPSIVGASWKEPFPGWIDNFNGP
SGLFIAAGKGILRTMRASNNALADLVPVDVVVNTSLAAAWYSGVNRPRNI
MVYNCTTGSTNPFHWGEVEYHVISTFKRNPLEQAFRRPNVNLTSNHLLYH
YWIAVSHKAPAFLYDIYLRMTGRSPRMMKTITRLHKAMVFLEYFTSNSWV
WNTDNVNMLMNQLMPEDKKTFNIDVRQLHWAEYIENYCMGTKKYVLNEEM
SGLPAARKHLNKLRNIRYGFNTILVILIWRIFIARSQMARNIWYFVVSLC
YKFLSYFRASSTMRY

A suitable enzyme for converting a C18 aldehyde to a C17 alkane is Anabidopsis thaliana gl1 homolog protein (Genbank accession no. U40489) as disclosed in Hansen et al., Plant Physiol. 113 (4):1091-1100 (1997), which is incorporated in its entirety by reference. The gl1 homolog protein has the following amino acid sequence (SEQ ID NO:8):

MATKPGVLTSWPWTPLGSFKYIVIAPWAVHSTYRFVTDDPEKRDLGYFLV
FPFLLFRILHNQVWISLSRYYTSSGKRRIVDKGIDFNQVDRETNWDDQIL
FNGVLFYIGINLLAEGKQLPWWRTDGVLMGALIHTGPVEFLYYWVHKALH
HHFLYSRYHSHHHSSIVTEPITSVIHPFAEHIAYFILFAIPLLTTLVTKT
ASIISFAGYIIYIDFMNNMGHCNFELIPKRLFHLFPPLKFLCYTPSYHSL
HHTQFRTNYSLFMPLYDYIYGTMDESTDTLYEKTLERGDDRVDVVHLTHL
TTPESIYHLRIGLPSFASYPFAYRWFMRLLWPFTSLSMIFTLFYARLFVA
ERNSFNKLNLQSWVIPRYNLQYLLKWRKEAINNMIEKAILEADKKGVKVL
SLGLMNQGEELNRNGEVYIHNHPDMKVRLVDGSRLAAAVVINSVPKATTS
VVMTGNLTKVAYTIASALCQRGVQVSTLRLDEYEKIRSCVPQECRDHLVY
LTSEALSSNKVWLVGEGTTREEQEKATKGTLFIPFSQFPLKQLRSDCIYH
TTPALIVPKSLVNVHSCENWLPRKAMSATRVAGILHALEGWETHECGTSL
LLSDLDKVWEACLSHGFQPLLLPHH

A suitable reductase is an enzyme capable of reducing C18 alcohol into C18 alkane. Such as a reductase should be found in Vibrio furnisii M1 as described in Park, J. Bacteriol. 187(4):1426-1429, 2005, which is incorporated in its entirety by reference.

The enzymes described herein can be readily replaced using a homologous enzyme thereof. A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme retains amino acids residues that are recognized as conserved for the enzyme. The homologous enzyme may have non-conserved amino acid residues replaced or found to be of a different amino acid, or amino acid(s) inserted or deleted, but which do not affect or has insignificant effect on the enzymatic activity of the homologous enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xho1, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host cell simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. Examples include lactose promoters (Lad repressor protein changes conformation when contacted with lactose, thereby preventing the LacI repressor protein from binding to the operator) and tryptophan promoters (when complexed with tryptophan, TrpR repressor protein has a conformation that binds the operator; in the absence of tryptophan, the TrpR repressor protein has a conformation that does not bind to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and λ phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

The host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of acetyl-CoA, the starting material for butyryl-CoA, C10-CoA, C14-CoA, C18-CoA, C18 aldehyde, C18 alcohol, C18 alkane and C17 alkane synthesis, is ensured. When added, the intermediate is present in an excess amount in the culture medium.

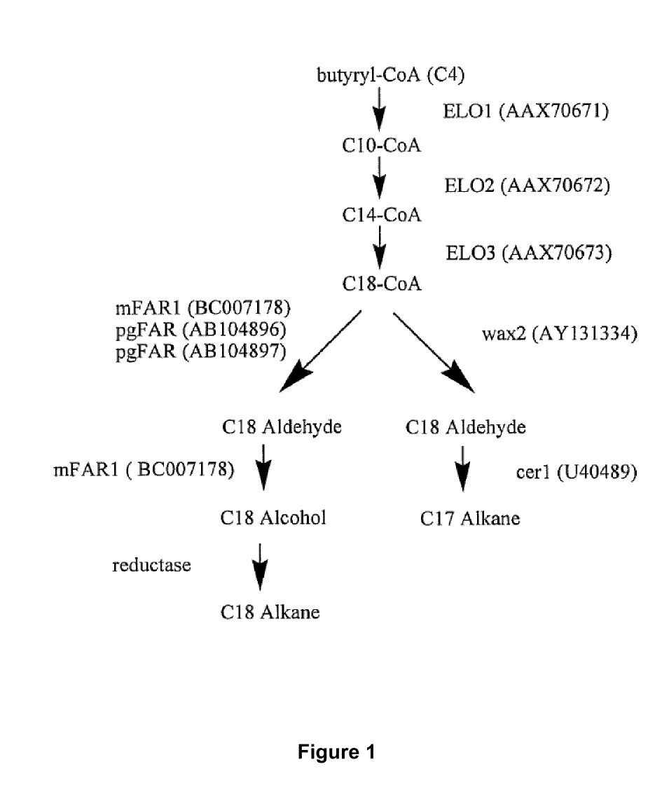

As the host cell grows and/or multiplies, expression of the enzymes necessary for producing butyryl-CoA, C10-CoA, C14-CoA, C18-CoA, C18 aldehyde, C18 alcohol, C18 alkane and C17 alkane is effected. Once expressed, the enzymes catalyze the steps necessary for carrying out the enzymatic steps shown in FIGS. 1 and 2. If an intermediate has been introduced, the expressed enzymes catalyze those steps necessary to convert the intermediate into the respective fatty acid derived compounds. Any means for recovering the C10-CoA, C14-CoA, C18-CoA, C18 aldehyde, C18 alcohol, C18 alkane and C17 alkane from the host cell may be used. For example, the host cell may be harvested and subjected to hypotonic conditions, thereby lysing the cells. The lysate may then be centrifuged and the supernatant subjected to high performance liquid chromatography (HPLC) or gas chromatography (GC).

### Host Cells

The host cells of the present invention are genetically modified in that heterologous nucleic acid have been introduced into the host cells, and as such the genetically modified host cells do not occur in nature. The suitable host cell is one capable of expressing a nucleic acid construct encoding an enzyme capable of catalyzing a desired biosynthetic reaction in order to produce the enzyme for producing the desired fatty acid molecule. Such enzymes are described herein. In some embodiments, the host cell naturally produces any of the precursors, as shown in FIGS. 1 and 2, for the production of the fatty acid derived compounds. These genes encoding the desired enzymes may be heterologous to the host cell or these genes may be native to the host cell but are operatively linked to heterologous promoters and/or control regions which result in the higher expression of the gene(s) in the host cell. In other embodiments, the host cell does not naturally produce butyryl-CoA, and comprises heterologous nucleic acid constructs capable of expressing one or more genes necessary for producing butyryl-CoA.

Each of the desired enzyme capable of catalyzing the desired reaction can be native or heterologous to the host cell. Where the enzyme is native to the host cell, the host cell is optionally genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

The genetically modified host cell can further comprise a genetic modification whereby the host cell is modified by the increased expression of one or more genes involved in the production of fatty acid compounds; such that the production of fatty acid compounds by the host cell is increased. Such genes encode following proteins: acetyl carboxylase (ACC), cytosolic thiosterase (teas), and acyl-carrier protein (AcpP). In some embodiments, the genetically modified host cell may be modified to produces higher levels of cytosolic acetyl-coA. Thus, in some embodiments, a host cell may comprise a modification to express, or increase expression of a protein such as ATP citrate lyase. For example, Saccharomyces cerevisiae has little ATP citrate lyase and can be engineered to express ATP citrate lyase by introducing an expression vector encoding ATP citrate lyase into the yeast cells.

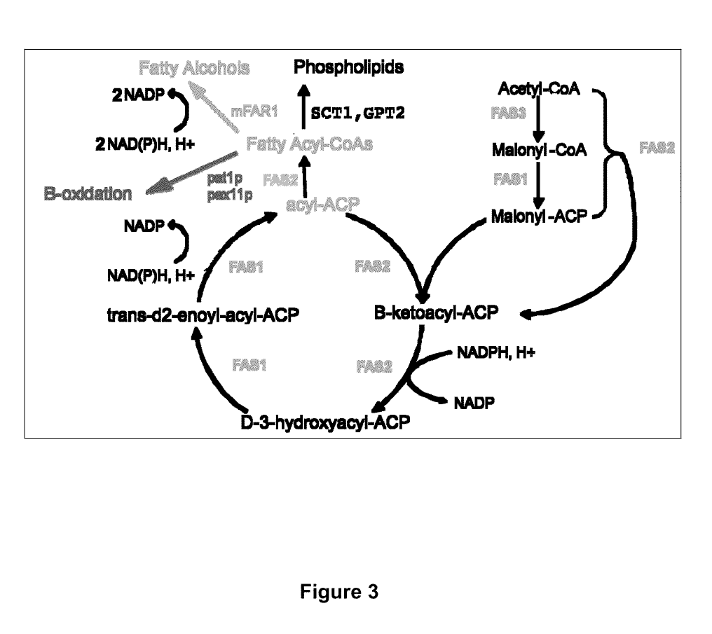

In some embodiments, a genetically modified host cell can be modified to increase expression of a Type I (eukaryotic) or Type II (prokaryotic) fatty acid synthase (FAS) gene. For example, a yeast host cell may be modified to express a FAS gene as shown in FIG. 3. Fatty acid synthase proteins are known in the art. FAS3 catalyzes the first committed step in fatty acid biosynthesis and in yeast is encoded by a 6.7 kb gene and contains two enzymatic domains: biotin carboxylase, and biotin carboxyltransferase. FAS2 is encoded, in yeast, by a 5.7 kb gene and contains four domains: an acyl-carrier protein, beta-ketoacyl reductase, beta-ketoacyl synthase, and phosphopantetheinyl transferase (PPT). FAS1 is encoded, in yeast, by a 6.2 kb gene and contains five domains: acetyltransacylase, dehydratase, enoyl reductase, malonyl transacylase, and palmitoyl transacylase. FAS1 and FAS2 complex to form a heterododecamer, containing six each of FAS1 and FAS2 subunits (Lomakin et al., Cell 129:319-322, 2007).

The genetically modified host cell can further comprise a genetic modification whereby the host cell is modified by the decreased or lack of expression of one or more genes encoding proteins involved in the storage and/or metabolism of fatty acid compounds; such that the storage and/or metabolism of fatty acid compounds by the host cell is decreased. Such genes include the following: the are1, are2, dga1, and/or lro1 genes. In some embodiments, the host cell is modified by the decreased or lack of expression of genes that are involved in the β-oxidation of fatty acids. For example, in yeast such, e.g., Saccharomyces cerevisiae, β-oxidation occurs in the peroxisome. Genes such as pat1 and pex11 are peroxisomal proteins involved in degradation of long-chain and medium-chain fatty acids, respectively. Accordingly, a host cell may be modified to delete pat1 and/or pex11, or otherwise decrease expression of the Pat1 and/or Pex11 proteins.

The genetically modified host cell can further comprise a genetic modification whereby the host cell is modified to express or have increased expression of an ABC transporter that is capable of exporting or increasing the export of any of the fatty acid derived compounds from the host cell. Such an ABC transporter is the plant Cer5.

Any prokaryotic or eukaryotic host cell may be used in the present method so long as it remains viable after being transformed with a sequence of nucleic acids. Generally, although not necessarily, the host microorganism is bacterial. In some embodiments, the bacteria is a cyanobacteria. Examples of bacterial host cells include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, Synechococcus, Synechocystis, and Paracoccus taxonomical classes. Preferably, the host cell is not adversely affected by the transduction of the necessary nucleic acid sequences, the subsequent expression of the proteins (i.e., enzymes), or the resulting intermediates required for carrying out the steps associated with the mevalonate pathway. For example, it is preferred that minimal “cross-talk” (i.e., interference) occur between the host cell's own metabolic processes and those processes involved with the mevalonate pathway.

Suitable eukaryotic cells include, but are not limited to, fungal, insect or mammalian cells. Suitable fungal cells are yeast cells, such as yeast cells of the Saccharomyces genus. In some embodiments the eukaryotic cell is an algae, e.g., Chlamydomonas reinhardtii, Scenedesmus obliquus, Chlorella vulgaris or Dunaliella salina.

The present invention provides for an isolated fatty acid derived compound produced from the method of the present invention. Isolating the fatty acid derived compound involves the separating at least part or all of the host cells, and parts thereof, from which the fatty acid derived compound was produced, from the isolated fatty acid derived compound. The isolated fatty acid derived compound may be free or essentially free of impurities formed from at least part or all of the host cells, and parts thereof. The isolated fatty acid derived compound is essentially free of these impurities when the amount and properties of the impurities do not interfere in the use of the fatty acid derived compound as a fuel, such as a fuel in a combustion reaction. These host cells are specifically cells that do not in nature produce the desired fatty acid derived compound.

The present invention also provides for a combustible composition comprising an isolated fatty acid derived compound and cellular components, wherein the cellular components do not substantially interfere in the combustion of the composition. The cellular components include whole cells or parts thereof. The cellular components are derived from host cells which produced the fatty acid derived compound.

The fatty acid derived compound of the present invention are useful as fuels as chemical source of energy that can be used as an alternative to petroleum derived fuels, ethanol and the like. The fatty acid derived compounds of the present invention are also useful in the synthesis of alkanes, alcohols, and esters of various for use as a renewable fuel. In addition, the fatty acid derived compounds can also be as precursors in the synthesis of therapeutics, or high-value oils, such as a cocoa butter equivalent. The fatty acid derived compounds are also useful in the production of the class of eicosanoids or related molecules, which have therapeutic related applications.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Production of C10-CoA, C14-CoA and C18-CoA in an E. coli host cell

Synthesis of butyryl-CoA has been shown in E. coli (Kennedy et al. Biochemistry, 42 (48):14342-14348 (2003), which is incorporated in its entirety by reference). Primers can be designed to PCR Clostridium acetobutylicum ATCC824 butyryl-CoA biosynthetic genes from the Clostridium acetobutylicum ATCC824 genomic DNA and have the genes cloned into a suitable E. coli expression vector. The resultant plasmid is introduced into an E. coli host cell. The resulting transformant, when cultured in a suitable medium, such as Luria broth (LB) medium, at 37° C. with the appropriate antibiotics to maintain the plasmids, is capable of producing butyryl-CoA.

The desired genes encoding Trypanosoma brucei elongases (ELO1, ELO2, and ELO3) can be PCRed from Trypanosoma brucei and cloned into a suitable E. coli expression vector, such that all three elongase genes are capable of expression in E. coli. Plasmids can also be designed and constructed that express ELO1 only or ELO1 and ELO2. Each plasmid is then separately transformed into the butyryl-CoA producing E. coli host cell described above to give rise to three different transformants.

Each resulting transformant is cultured in a suitable medium, such as LB medium at 37° C. with the appropriate antibiotics to maintain the plasmids. The enzymes are induced using the appropriate inducers, such as IPTG or propionate, and incubated at 30° C. for 3-7 days. The induction of the enzymes results in the production of the appropriate CoA compound.

The transformant which expresses ELO1 is capable of producing C10-CoA. The transformant which expresses ELO1 and ELO2 is capable of producing C10-CoA and C14-CoA. The transformant which expresses ELO1, ELO2, and ELO3 is capable of producing C10-CoA C14-CoA, and C18-CoA.

The C10-CoA C14-CoA, and C18-CoA produced can be purified and analyzed using a gas chromatography-mass spectrometer (GC-MS).

### Example 2

### Production of C18 aldehyde in an E. coli host cell

Primers can be designed to PCR the gene encoding Arabidopsis thaliana cuticle protein (WAX2) from Arabidopsis thaliana genomic DNA and have the gene cloned into a suitable E. coli expression vector. Alternatively, primers can be designed to PCR the gene encoding Bombyx mori fatty-acyl reductase (FAR) from Bombyx mori genomic DNA and have the gene cloned into a suitable E. coli expression vector.

Either of the resultant plasmid is introduced into the E. coli host cell of Example 1, which is capable of producing C18-CoA. Each resulting transformant is cultured in a suitable medium, such as LB medium at 37° C. with the appropriate antibiotics to maintain the plasmids. The enzymes are induced using the appropriate inducers, such as IPTG or propionate, and incubated at 30° C. for 3-7 days. The induction of the enzymes results in the production of C18 aldehyde. The C18 aldehyde produced can be purified and analyzed using a gas chromatography-mass spectrometer (GC-MS).

### Example 3

### Production of C18 aldehyde and C18 alcohol in an E. coli host cell

Primers can be designed to PCR the gene encoding Mus musculus male sterility domain containing 2 protein (FART) from Mus musculus genomic DNA and have the gene cloned into a suitable E. coli expression vector. The resultant plasmid is introduced into the E. coli host cell of Example 1, which is capable of producing C18-CoA. Each resulting transformant is cultured in a suitable medium, such as LB medium at 37° C. with the appropriate antibiotics to maintain the plasmids. The enzymes are induced using the appropriate inducers, such as IPTG or propionate, and incubated at 30° C. for 3-7 days. The induction of the enzymes results in the production of C18 aldehyde and C18 alcohol. The C18 aldehyde and C18 alcohol produced can be purified and analyzed using a gas chromatography-mass spectrometer (GC-MS).

### Example 4

### Production of C17 alkane in an E. coli host cell

Primers can be designed to PCR the gene encoding Arabidopsis thaliana gl1 homolog protein from Arabidopsis thaliana genomic DNA and have the gene cloned into a suitable E. coli expression vector. The resulting plasmid is introduced into the E. coli host cell of Example 1 which expresses WAX2, which is capable of producing C18 aldehyde. Each resulting transformant is cultured in a suitable medium, such as LB medium at 37° C. with the appropriate antibiotics to maintain the plasmids. The enzymes are induced using the appropriate inducers, such as IPTG or propionate, and incubated at 30° C. for 3-7 days. The induction of the enzymes results in the production of C18 aldehyde and C17 alkane. The C18 aldehyde and C17 alkane produced can be purified and analyzed using a gas chromatography-mass spectrometer (GC-MS).

### Example 5

### Production of C18 alkane in an E. coli host cell

The gene encoding a suitable reductase can be cloned by PCR and inserted into a suitable E. coli expression vector. The resulting plasmid is introduced into the E. coli host cell of Example 3 which expresses musculus male sterility domain containing 2 protein, which is capable of producing C18 alcohol. Each resulting transformant is cultured in a suitable medium, such as LB medium at 37° C. with the appropriate antibiotics to maintain the plasmids. The enzymes are induced using the appropriate inducers, such as IPTG or propionate, and incubated at 30° C. for 3-7 days. The induction of the enzymes results in the production of C18 alkane. The C18 alkane produced can be purified and analyzed using a gas chromatography-mass spectrometer (GC-MS).

### Example 6

### Increase production of fatty acids in an E. coli host cell

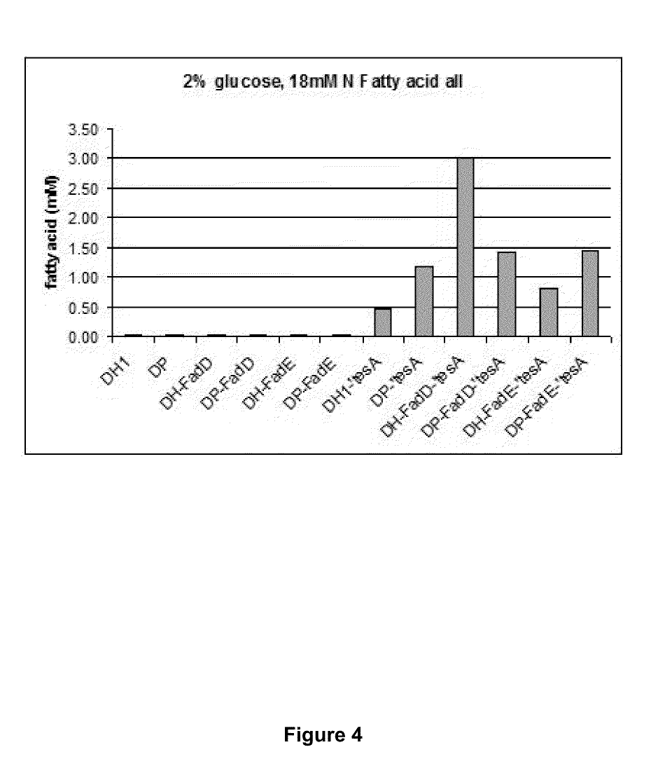

LtesA, a cytoxolic fatty acyl-coa/acp thioesterase (it lacks the leader sequence) was overexpressed in E. coli host cells that comprise various gene deletions that increase metabolic flux to fatty acid metabolism. The knockout backgrounds are as follows: DH1=wild type E. coli; DP=acetate knock-out; DH1 FadD=FadD knock-out; DP fadD=fadD knock-out in DP, DP sucA=sucA knock-out in DP. The Fad proteins are involved in the transport, activation and β-oxidation of fatty acids. The results (FIG. 4) obtained with limited Nitrogen, 2% glucose show that overexpression of LtesA in E. coli host cells that have increased metabolic flux to fatty acid metabolism increases production of fatty acids.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for producing a C18 aldehyde in a genetically modified host cell, the method comprising:
(a) culturing a genetically modified host cell under a suitable condition, wherein the genetically modified host cell comprises a first enzyme capable of converting a C18-CoA to a C18 aldehyde and optionally a C18 alcohol, and optionally a second enzyme capable of converting the C18 aldehyde to a C17 alkane or a third enzyme capable of converting the C18 alcohol to a C18 alkane,
such that the culturing results in the genetically modified host cell producing the C18 aldehyde, and optionally the C17 alkane, the C18 alcohol, or C18 alkane, or a combination thereof.

2. The method of claim 1, wherein the genetically modified host cell comprises at least one enzyme selected from the group consisting of Trypanasoma ELO1, ELO2, and ELO3 enzymes.

3. The method of claim 2. wherein the genetically modified host cell comprises a nucleic acid construct that encodes an enzyme that synthesizes butyryl-CoA from acetyl-CoA.

4. The method of claim 1, wherein the genetically modified host cell comprises a first nucleic acid construct encoding the first enzyme, and optionally a second nucleic acid construct encoding the second enzyme or third enzyme, and the culturing results in the expression of the first enzyme, and optionally the second enzyme or the third enzyme.

5. The method of claim 4, further comprising the step of: introducing the first nucleic acid construct, and optionally a second nucleic acid construct, into the genetically modified host cell, wherein the introducing step is prior the culturing step.

6. The method of claim 1, further comprising the step of:
(b) recovering the produced C18 aldehyde, or optionally the C17 alkane, the C18 alcohol, or the C18 alkane, or a combination thereof, wherein the recovering step is concurrent or subsequent to the culturing step.

7. The method of claim 1, wherein the first enzyme is Arabidopsis thaliana cuticle protein (WAX2), or Bombyx mori fatty-acyl reductase (FAR), or a homologous enzyme thereof, and the culturing results in the genetically modified host cell producing the C18 aldehyde.

8. The method of claim 1, wherein the first enzyme is Mus musculus male sterility domain containing 2 protein, or a homologous enzyme thereof, and the culturing results in the genetically modified host cell producing the C18 aldehyde and the C18 alcohol.

9. The method of claim 1, wherein the second enzyme is Arabidopsis thaliana gl1 homolog protein, or a homologous enzyme thereof, and the culturing results in the genetically modified host cell producing the C18 aldehyde and the C17 alkane.

10. The method of claim 1, wherein the third enzyme is a reductase, or a homologous enzyme thereof, and the culturing results in the genetically modified host cell producing the C18 aldehyde, C18 alcohol, and the C18 alkane.

11. The method of claim 1, wherein the host cell is a eubacteria.

12. The method of claim 11, wherein the host cell is one selected from the Escherichia, Enterobacter, Azotobacter, Erwinia, Bacillus, Pseudomonas, Klebsielia, Proteus, Salmonella, Serratia, Shigella, Rhizobia, Vitreoscilla, Synechococcus, Synechocystis, and Paracoccus taxonomical classes.

13. The method of claim 12, wherein the host cell is Escherichia coli.

14. The method of claim 1, wherein the host cell is an algal, fungal, insect or mammalian cell line.

15. The method of claim 14, wherein the host cell is a yeast.

16. The method of claim 15, wherein the host cell is Saccharomyces cerevisiae.

17. The method of claim 1, wherein the host cell further comprises a genetic modification whereby the expression of one or more genes involved in the production of fatty acid compounds is increased.

18. The method of claim 17, wherein the one or more genes involved in the production of fatty acid compounds are genes that encode acetyl carboxylase (ACC), cytosolic thiosterase (teas), or acyl-carrier protein (AcpP).

19. The method of claim 1, wherein the host cell further comprises a genetic modification whereby the expression of one or more genes encoding proteins involved in the storage or metabolism of fatty acid compounds is decreased or is not expressed.

20. The method of claim 19, wherein the one or more genes encoding proteins involved in the storage or metabolism of fatty acid compounds are the are1, are2, dga1, or lro1 genes.

21. The method of claim 19, wherein the one or more genes encoding proteins involve din the storage or metabolism of fatty acid compounds are the pat1 or pex11 genes.

22. The method of claim 1, wherein the host cell further comprises a genetic modification whereby the expression of an ABC transporter is increased.

23. The method of claim 22, wherein the ABC transporter is a plant Cer5.

24. A genetically modified host cell comprising a first nucleic acid construct encoding a first enzyme capable of converting a C18-CoA to a C18 aldehyde and optionally a C18 alcohol, and optionally a second enzyme capable of converting the C18 aldehyde to a C17 alkane or a third enzyme capable of converting the C18 alcohol to a C18 alkane, which under a suitable condition produces the C18 aldehyde, and optionally the C17 alkane, the C18 alcohol, or C18 alkane, or a combination thereof.

25. The host cell of claim 24, wherein the host cell prior to genetic modification does not produce C18-CoA, C18 aldehyde, and optionally the C17 alkane, the C18 alcohol, and the C18 alkane.

26. The host cell of claim 24, wherein the host cell further comprises a genetic modification whereby the expression of one or more genes involved in the production of fatty acid compounds is increased.

27. The host cell of claim 26, wherein the one or more genes involved in the production of fatty acid compounds are genes that encode acetyl carboxylase (ACC), cytosolic thiosterase (teas), or acyl-carrier protein (AcpP).

28. The host cell of claim 24, wherein the host cell further comprises a genetic modification whereby the expression of one or more genes encoding proteins involved in the storage or metabolism of fatty acid compounds is decreased or is not expressed.

29. The host cell of claim 28, wherein the one or more genes encoding proteins involved in the storage or metabolism of fatty acid compounds are the are1, are2, dga1, or lro1 genes.

30. The host cell of claim 28, wherein the one or more genes encoding proteins involved in the storage or metabolism of fatty acid compounds are the pat1 or pex11 genes.

31. The host cell of claim 24, wherein the host cell further comprises a genetic modification whereby the expression of an ABC transporter is increased.

32. The host cell of claim 31, wherein the ABC transporter is a plant Cer5.

33. A genetically modified host cell that comprises one or more nucleic acid constructs, wherein the one or more nucleic acid constructs encode a first enzyme capable of converting butyryl-CoA to C10-CoA, a second enzyme capable of converting C10-CoA to C14-CoA; and a third enzyme capable of converting the C14-CoA to C18-CoA.

34. The host cell of claim 33, wherein the first enzyme is Trypanosoma brucei ELO1, the second enzyme is Trypanosoma brucei ELO2, and the third enzyme is Trypanosoma brucei ELO3.

35. The host cell of claim 33, wherein the first, second, and third enzyme are encoded by a single plasmid.

36. The host cell of claim 33, wherein the genetically modified host cell comprises a nucleic acid construct that encodes an enzyme that synthesizes butyryl-CoA from acetyl-CoA.

37. A combustible composition comprising an isolated C18 aldehyde, C17 alkane, C18 alcohol, or C18 alkane and cellular components, wherein the cellular components do not substantially interfere in the combustion of the composition.

38. The composition of claim 37 wherein the cellular components are of cells which do not naturally produce the C18 aldehyde, C17 alkane, C18 alcohol, or C18 alkane.

39. A method for producing a C18-CoA in a genetically modified host cell, the method comprising:
(a) culturing a genetically modified host cell under a suitable condition, wherein the genetically modified host cell comprises a first enzyme capable of converting butyryl-CoA to C10-CoA, a second enzyme capable of converting C10-CoA to C14-CoA; and a third enzyme capable of converting the C14-CoA to C18-CoA,
such that the culturing results in the genetically modified host cell producing the C18-CoA.

40. The method of claim 39, wherein the first enzyme is Trypanosoma brucei ELO1, the second enzyme is Trypanosoma brucei ELO2, and the third enzyme is Trypanosoma brucei ELO3.

41. The method of claim 39, wherein the genetically modified host cell comprises a nucleic acid construct that encodes an enzyme that synthesizes butyryl-CoA from acetyl-CoA.


SEQUENCE LISTING (8 sequences)

SEQ ID NO: 1 - protein, 305 aa, Trypanosoma brucei
  fatty acid elongase (ELO1)
MFFTPPQLQKLEQDWNGLAVRDWMIANVDVVLYISFLYLGFVFIGPKLFAKLVGTNPAAAAAGARSADGTGSPIVRRSMVVWNLALSIFSIFGTSTVTPVLLRNLANKGFYGATCDFKETEFYTTNVGFWMGIFALSKIPELVDTIFLVLQGKQELPFLHWYHHVTVLLFSWHTYCVGSSAYIWVAAMNYSVHSVMYLYFALAALGYKRVVRPLAPYITIIQILQMVVGCYVTIFALQELHGEGGRGCGVSPANMRIQLVMYASYLYLFSKMFVASYIRPPKRPTVGGPSSTAGVSNGSVEKKVK

SEQ ID NO: 2 - protein, 271 aa, Trypanosoma brucei
  fatty acid elongase (ELO2)
MFPYVTDYSGFAIRKWMIDNVDVAGFLCLLYLGLVWKGPGVVKSLREKNLINATLLQGVFIMWNLFLSTFSVIGMIVVVPAAIAHISNKGLVPALCERDVNMIYDSPVGFWVGVFALSKIPELFDTVLLVLQGKQPPFLHWYHHTTVLIFSWQSYCEGSSTIFVFVAMNLTVHAVMYFYFAMCASGFKAIMRTIAPVITIMQILQMIVGSAVTMYSAYVLYNPQPDGPQTCDVTKASARMGVVMYLSYLYLFAALFVESYLKPKKRTEKSK

SEQ ID NO: 3 - protein, 301 aa, Trypanosoma brucei
  fatty acid elongase (ELO3)
MLMNFGGSYDAYINNFQGTFLAEWMLDHPSVPYIAGVMYLILVLYVPKSIMASQPPLNLRAANIVWNLFLTLFSMCGAYYTVPYLVKAFMNPEIVMAASGIKLDANTSPIITHSGFYTTTCALADSFYFNGDVGFWVALFALSKIPEMIDTAFLVFQKKPVIFLHWYHHLTVMLFCWFAYVQKISSGLWFASMNYSVHSIMYLYYFVCACGHRRLVRPFAPIITFVQIFQMVVGTIVVCYTYTVKHVLGRSCTVTDFSLHTGLVMYVSYLLLFSQLFYRSYLSPRDKASIPHVAAEIKKKE

SEQ ID NO: 4 - protein, 632 aa, Arabidopsis thaliana
  cuticle protein (WAX2)
MVAFLSAWPWENFGNLKYLLYAPLAAQVVYSWVYEEDISKVLWCIHILIICGLKALVHELWSVFNNMLFVTRTLRINPKGIDFKQIDHEWHWDNYIILQAIIVSLICYMSPPLMMMINSLPLWNTKGLIALIVLHVTFSEPLYYFLHRSFHRNNYFFTHYHSFHHSSPVPHPMTAGNATLLENIILCVVAGVPLIGCCLFGVGSLSAIYGYAVMFDFMRCLGHCNVEIFSHKLFEILPVLRYLIYTPTYHSLHHQEMGTNFCLFMPLFDVLGDTQNPNSWELQKKIRLSAGERKRVPEFVFLAHGVDVMSAMHAPFVFRSFASMPYTTRIFLLPMWPFTFCVMLGMWAWSKTFLFSFYTLRNNLCQTWGVPRFGFQYFLPFATKGINDQIEAAILRADKIGVKVISLAALNKNEALNGGGTLFVNKHPDLRVRVVHGNTLTAAVILYEIPKDVNEVFLTGATSKLGRAIALYLCRRGVRVLMLTLSMERFQKIQKEAPVEFQNNLVQVTKYNAAQHCKTWIVGKWLTPREQSWAPAGTHFHQFVVPPILKFRRNCTYGDLAAMKLPKDVEGLGTCEYTMERGVVHACHAGGVVHMLEGWKHHEVGAIDVDRIDLVWEAAMKYGLSAVSSLTN

SEQ ID NO: 5 - protein, 460 aa, Bombyx mori
  first fatty-acyl reductase (FAR)
MSHNGTLDEHYQTVREFYDGKSVFITGATGFLGKAYVEKLAYSCPGIVSIYILIRDKKGSNTEERMRKYLDQPIFSRIKYEHPEYFKKIIPISGDITAPKLGLCDEERNILINEVSIVIHSAASVKLNDHLKFTLNTNVGGTMKVLELVKEMKNLAMFVYVSTAYSNTSQRILEEKLYPQSLNLNEIQKFAEEHYILGKDNDEMIKFIGNHPNTYAYTKALAENLVAEEHGEIPTIIIRPSIITASAEEPVRGFVDSWSGATAMAAFALKGWNNIMYSTGEENIDLIPLDYVVNLTLVAIAKYKPTKEVTVYHVTTSDLNPISIRRIFIKLSEFASKNPTSNAAPFAATTLLTKQKPLIKLVTFLMQTTPAFLADLWMKTQRKEAKFVKQHNLVVRSRDQLEFFTSQSWLLRCERARVLSAALSDSDRAVFRCDPSTIDWDQYLPIYFEGINKHLFKNKL

SEQ ID NO: 6 - protein, 460 aa, Bombyx mori
  second fatty-acyl reductase (FAR)
MSHNGTLDEHYQTVSEFYDGKSVFITGATGFLGKAYVEKLAYSCPGIVSIYILIRNKKGSNTEERMRKYLDQPIFSRIKYEHPEYFKKIIPISGDIAAPKLGLCDEERNILINEVSIVIHSAASVKLNDHLKFTLNTNVGGTMKVLELVKEMKNLAMFVYVSTAYSNTSQRILEEKLYPQSLNLSEIQKFAEEHYILGKDDDEMIKFIGNHPNTYAYTKALAENLVAEEHGEIPTIIIRPSIITASAEEPVRGFVDSWSGATAMAASTLKGWNYIMYSTGEENIDLIPLDYVVNLTLVAIAKNKPTKEVTVYHVTTSDLNPISIRRIFIKLSEFASKNPTSNAAPFAATTLLTKQKPLIKLVTFLMQTTPAFLADFWMKTQRKEAKFVKQHNLVVRSRDQLEFFPSQSWLLRCERARVLSAGLGDSGRAVFRCDPSPIDWDQYLPIYFEGINKHLFKNKF

SEQ ID NO: 7 - protein, 515 aa, Mus musculus
  male sterility domain containing 2 protein, fatty acyl CoA reductase 1 (FAR1)
MVSIPEYYEGKNILLTGATGFLGKVLLEKLLRSCPRVNSVYVLVRQKAGQTPQERVEEILSSKLFDRLRDENPDFREKIIAINSELTQPKLALSEEDKEIIIDSTNVIFHCAATVRFNENLRDAVQLNVIATRQLILLAQQMKNLEVFMHVSTAYAYCNRKHIDEVVYPPPVDPKKLIDSLEWMDDGLVNDITPKLIGDRPNTYIYTKALAEYVVQQEGAKLNVAIVRPSIVGASWKEPFPGWIDNFNGPSGLFIAAGKGILRTMRASNNALADLVPVDVVVNTSLAAAWYSGVNRPRNIMVYNCTTGSTNPFHWGEVEYHVISTFKRNPLEQAFRRPNVNLTSNHLLYHYWIAVSHKAPAFLYDIYLRMTGRSPRMMKTITRLHKAMVFLEYFTSNSWVWNTDNVNMLMNQLNPEDKKTFNIDVRQLHWAEYIENYCMGTKKYVLNEEMSGLPAARKHLNKLRNIRYGFNTILVILIWRIFIARSQMARNIWYFVVSLCYKFLSYFRASSTMRY

SEQ ID NO: 8 - protein, 625 aa, Arabidopsis thaliana
  maize gl1 homolog protein
MATKPGVLTDWPWTPLGSFKYIVIAPWAVHSTYRFVTDDPEKRDLGYFLVFPFLLFRILHNQVWISLSRYYTSSGKRRIVDKGIDFNQVDRETNWDDQILFNGVLFYIGINLLAEGKQLPWWRTDGVLMGALIHTGPVEFLYYWVHKALHHHFLYSRYHSHHHSSIVTEPITSVIHPFAEHIAYFILFAIPLLTTLVTKTASIISFAGYIIYIDFMNNMGHCNFELIPKRLFHLFPPLKFLCYTPSYHSLHHTQFRTNYSLFMPLYDYIYGTMDESTDTLYEKTLERGDDRVDVVHLTHLTTPESIYHLRIGLPSFASYPFAYRWFMRLLWPFTSLSMIFTLFYARLFVAERNSFNKLNLQSWVIPRYNLQYLLKWRKEAINNMIEKAILEADKKGVKVLSLGLMNQGEELNRNGEVYIHNHPDMKVRLVDGSRLAAAVVINSVPKATTSVVMTGNLTKVAYTIASALCQRGVQVSTLRLDEYEKIRSCVPQECRDHLVYLTSEALSSNKVWLVGEGTTREEQEKATKGTLFIPFSQFPLKQLRSDCIYHTTPALIVPKSLVNVHSCENWLPRKAMSATRVAGILHALEGWETHECGTSLLLSDLDKVWEACLSHGFQPLLLPHH